(19) United States
(12) Patent Application Publication
(10) Pub. No.: US 2019/0290759 A1
(43) Pub. Date: Sep. 26, 2019

(54) COMBINATION OF HDAC INHIBITOR AND ANTI-PD-L1 ANTIBODY FOR TREATMENT OF OVARIAN CANCER

(71) Applicants: Syndax Pharmaceuticals, Inc., Waltham, MA (US); MERCK PATENT GMBH, Darmstadt (DE)

(72) Inventors: Peter Ordentlich, Lexington, MA (US); Michael Meyers, Waltham, MA (US)

(21) Appl. No.: 16/067,020

(22) PCT Filed: Dec. 28, 2016
(86) PCT No.: PCT/US16/68836

§ 371 Date: Jun. 28, 2018

Related U.S. Application Data
(60) Provisional application No. 62/271,914, filed on Dec. 28, 2015.

(51) Int. Cl. A61K 39/395; C07K 16/28; A61K 31/4406; A61P 35/00

(52) U.S. Cl. CPC A61K 39/39566; C07K 16/2827; A61K 2039/505; A61P 35/00; C07K 2317/21; A61K 31/4406

(57) ABSTRACT

Described herein are methods for the treatment of heavily pre-treated recurrent ovarian cancer. In particular, methods are provided for the treatment of heavily pre-treated recurrent ovarian cancer with a combination of entinostat and an anti-PD-L1 antibody.

### RELATED APPLICATIONS

This application is a national stage application, filed under 35 U.S.C. § 371, of PCT Application No. PCT/US2016/068836 filed Dec. 28, 2016, which claims priority to, and the benefit of, U.S. Provisional Application No. 62/271,914, filed Dec. 28, 2015, the entire content of each of which is incorporated herein by reference in their entireties.

### SUMMARY

Provided herein in one embodiment is a method of treating cancer, comprising administering to a patient a combination comprising entinostat and an anti-PD-L1 antibody, wherein the cancer is ovarian cancer. In some embodiments, the ovarian cancer is refractory or recurrent epithelial ovarian cancer. In some embodiments, the anti PD-L1 antibody is avelumab. In some embodiments, the ovarian cancer is heavily pretreated recurrent ovarian cancer. In some embodiments, the heavily pretreated recurrent ovarian cancer is epithelial ovarian carcinoma, fallopian tube cancer, or primary peritoneal carcinoma. In some embodiments, the heavily pretreated recurrent ovarian cancer is epithelial ovarian carcinoma. In some embodiments, the patient has received at least one round of a prior therapy. In some embodiments, the patient has received at least three rounds of a prior therapy. In some embodiments, the prior therapy is platinum based chemotherapy. In some embodiments, the patient has a relapse of ovarian cancer within six months after the last round of platinum based chemotherapy. In some embodiments, entinostat and anti-PD-L1 antibody are administered sequentially in either order or simultaneously. In some embodiments, the anti-PD-L1 antibody is administered by intravenous infusion. In some embodiments, the anti-PD-L1 antibody is administered once every two weeks during the treatment cycle, at a dose of 10 mg/kg. In some embodiments, the entinostat is administered orally. In some embodiments, the entinostat is administered once every week during the treatment cycle, at a dose of 3 mg. In some embodiments, the entinostat is administered once every week during the treatment cycle, at a dose of 5 mg. In some embodiments, the entinostat is administered once every two weeks during the treatment cycle, at a dose of 10 mg. In some embodiments, the entinostat is administered first. In some embodiments, the entinostat is administered weekly. In some embodiments, the entinostat is administered every two weeks. In some embodiments, the entinostat is administered at a dose of 5 mg. In some embodiments, entinostat and anti-PD-L1 antibody are administered simultaneously.

Provided herein in one embodiment is a kit for treating heavily pre-treated recurrent ovarian cancer comprising a combination of entinostat and an anti-PD-L1 antibody. In some embodiments, the anti-PD-L1 antibody is avelumab.

All publications, patents, and patent applications described in this specification are herein incorporated by reference to the same extent as if each individual publication, patent, or patent application was specifically and individually indicated to be incorporated by reference. Citation of publications and patent documents is not intended as an admission that any is pertinent prior art, nor does it constitute any admission as to the contents or date of the same.

### BRIEF DESCRIPTION OF THE DRAWINGS

FIG. 1 is a group of immunofluorescence microscopic images showing that vorinostat and entinostat modulate PD-L1 expression in human lung and prostate tumor xenografts.

FIG. 2 is a group of graphs showing that vorinostat and entinostat enhance avelumab-mediated ADCC of prostate and human lung carcinoma cells.

FIG. 3 is a pair of graphs showing that avelumab-mediated lysis of carcinoma cells is decreased by CD16 neutralization.

### DETAILED DESCRIPTION

Provided herein are methods of treating ovarian cancer based on the administration of an HDAC inhibitor and an anti PD-L1 antibody. The methods may further include treatments wherein the combination is supplemented with one or more therapeutic agents or therapies.

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

Cancer, tumors, tumor-related disorders, and neoplastic disease states are serious and often times life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

HDAC inhibitors are an emerging class of therapeutic agents that promote differentiation and apoptosis in hematologic and solid malignancies through chromatin remodeling and gene expression regulation. Several HDAC inhibitors have been identified including benzamides (entinostat), short-chain fatty acids (i.e., Sodium phenylbutyrate); hydroxamic acids (i.e., suberoylanilide hydroxamic acid and trichostatin A); cyclic tetrapeptides containing a 2-amino-8-oxo-9,10-epoxy-decanoyl moiety (i.e., trapoxin A) and cyclic peptides without the 2-amino-8-oxo-9,10-epoxy-decanoyl moiety (i.e., FK228). Entinostat is a benzamide HDAC inhibitor undergoing clinical investigation in multiple types of solid tumors and hematologic cancers. Entinostat is rapidly absorbed and has a half-life of about 100 hours and, importantly, changes in histone acetylation persist for several weeks following the administration of entinostat.

High expression of PD-1/PD-L1 on tumor cells has been found to correlate with poor prognosis and survival in various other solid tumor types. Without being bound by any theory it is contemplated that the PD-1/PD-L1 pathway plays a critical role in the tumor immune evasion and could be considered an attractive target for therapeutic intervention in several solid organ types.

Several PD-1 and PD-L1 antibodies are in clinical development. Overall, they have been reported to be well tolerated, with most not reaching dose-limiting toxicity in their phase I studies.

### Histone Deacetylase

The HDACs are a family including at least eighteen enzymes, grouped in three classes (Class I, II and III). Class I HDACs include, but are not limited to, HADCs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not interact with all three HDAC classes. HDAC Inhibitors

HDAC inhibitors can be classified broadly into pan HDAC inhibitors and selective HDAC inhibitors. Although there is a large structural diversity of known HDAC inhibitors, they share common features: a part that interacts with the enzyme active site and a side-chain that sits inside the channel leading to the active site. This can be seen with the hydroxamates such as SAHA, where the hydroxamate group is believed to interact with the active site. In the case of the depsipeptides, it is believed that an intracellular reduction of the disulphide bond creates a free thiol group (which interacts with the active site) attached to a 4-carbon alkenyl chain. A difference between the HDAC inhibitors is in the way that they interact with the rim of the HDAC channel, which is at the opposite end of the channel to the active site. It is this interaction, between the HDAC inhibitor and the rim of the channel, which is believed to account, at least in part, for some observed differences in HDAC selectivity between pan-HDAC inhibitors, such as SAHA and selective HDAC inhibitors such as the depsipeptides. A particularly preferred HDAC inhibitor is entinostat. Entinostat has the chemical name N-(2-aminophenyl)-4-[N-(pyridine-3-yl)methoxycarbonylamino-methyl]-benzamide and the chemical structure shown below.

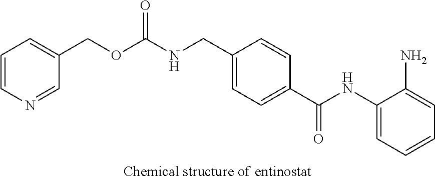 [SMILES: Nc1ccccc1NC(=O)c1ccc(CNC(=O)OCc2cccnc2)cc1]

### Programmed Cell Death-1 (PD-1)

PD-1 is a cell surface receptor that is a member of the CD28 family of T-cell regulators, within the immunoglobulin superfamily of receptors. The human PD-1 gene is located at chromosome 2q37, and the full-length PD-1 cDNA encodes a protein with 288 amino acid residues with 60% homology to murine PD-1. It is present on CD4− CD8− (double negative) thymocytes during thymic development and is expressed upon activation in mature hematopoietic cells such as T and B cells, NKT cells and monocytes after prolonged antigen exposure.

Without being bound by any theory, it is contemplated that binding of the ligand PD-L1 to PD-1 downregulates effector anti-tumor T-cell activity and facilitates immune evasion. This is supported by the finding of an association between PD-1/PD-L1 expression and poor prognosis in several tumor types including gastric, ovarian, lung and renal carcinomas. PD-1 has been reported to be predominantly expressed by tumor infiltrating T lymphocytes, in melanoma.

In vitro studies of PD-1 blockade by PD-1-specific antibody showed augmentation of cytotoxic T-cell responses to melanoma-specific antigens including increased frequencies of IFN-γ-secreting antigen-specific cells.

Without being bound by any theory, it is contemplated that targeting PD-1 may act as an effective therapeutic strategy for cancer.

The principal method for targeting PD-1 clinically has been through the development of genetically engineered monoclonal antibodies that inhibit either PD-1 or PD-L1 function.

PD-L1 has also been shown to bind to B7-1 (CD80), an interaction that also suppresses T-cell proliferation and cytokine production; however, the exact relative contributions of the PD-L1:PD-1 and PD-L1:B7-1 pathways in cancer remain unclear. The PD-1-targeting agents currently in development inhibit both pathways. However, as the binding sites for PD-1 and B7-1 are adjacent but not overlapping, agents that specifically target one or the other may potentially be developed.

Cancer cells drive high expression levels of PD-L1 on their surface, allowing activation of the inhibitory PD-1 receptor on any T cells that infiltrate the tumor microenvironment, effectively switching those cells off. Indeed, upregulation of PD-L1 expression levels has been demonstrated in many different cancer types (e.g., melanoma [40%-100%], NSCLC [35%-95%], and multiple myeloma [93%]), and high levels of PD-L1 expression have been linked to poor clinical outcomes. Furthermore, tumor-infiltrating T cells have been shown to express significantly higher levels of PD-1 than T cells that infiltrate normal tissue. It is thought that the tumor microenvironment may secrete pro-inflammatory cytokines, including interferon-gamma (IFNγ) to upregulate the expression of PD-1 on tumor-infiltrating T cells to ensure that they can respond to the high levels of PD-L1 expressed on the tumor.

### Avelumab

Avelumab (MSB0010718C) is a fully human anti-PD-L1 IgG1 antibody currently being investigated in clinical trials. In addition to disruption of immune suppressive signaling induced by the binding of PD-L1 on tumor cells with PD-1 on tumor infiltrating immune cells, avelumab is designed to mediate antibody dependent cellular cytotoxicity (ADCC). The ability of avelumab to induce lysis of human carcinoma cells has been assessed using whole peripheral blood mononuclear cells (PBMCs) or purified natural killer (NK) cells as effectors.

In a recent study (Kwong-Yok Tsang et al., Antibody dependent cellular cytotoxicity activity of a novel anti-PD-L1 antibody, avelumab (MSB0010718C), on human tumor cells, 2015 ASCO Annual Meeting, J Clin Oncol 33, 2015 (suppl; abstr 3038)), using PBMCs as effectors, avelumab was found to induce ADCC in 8 out of 18 human carcinoma cell lines. Furthermore, tumor cell lysis was found to positively correlate with the percentage of PD-L1 positive tumor cells. The percentage of PD-L1 positive tumor cells was reported as mean fluorescence intensity (MFI) determined using flow cytometry. Lysis was increased when NK cells were used as effectors. Pretreating tumor cell lines with IFN-γ increased PD-L1 expression, but augmented lysis in only 4 out of 10 cell lines. Preactivating NK cells with IL-12, however, increased lysis, suggesting a potential for synergy by combining avelumab with IL-12-based therapy. Little or no lysis was observed in NK-mediated ADCC assays vs whole PBMCs or dendritic cells isolated from PBMCs. A tumor cell line insensitive to lysis by CD8+T cells was lysed by ADCC using NK cells and avelumab. In conclusion, the study found that Avelumab induced lysis of many human tumor cell lines via ADCC and further clinical trials are necessary to determine whether the additional mechanism of inducing tumor lysis by ADCC will result in enhanced clinical activity compared with similar agents without ADCC activity.

### Ovarian Cancer

Ovarian cancer is the 8th most common cancer in women worldwide with estimated 225,500 new diagnoses per year and estimated 140,200 deaths per year.

There are three basic types of ovarian tumors: epithelial, germ cell, and stromal cell tumors. Epithelial tumors start from the cells that cover the outer surface of the ovary; most ovarian tumors are epithelial cell tumors. Germ cell tumors start from the cells that produce the eggs. Stromal tumors start from cells that hold the ovary together and make the female hormones. A significant risk factor for ovarian cancer includes deficiencies in DNA repair via homologous recombination, such as mutations in the BRCA1 or BRCA2 gene. Those genes were originally identified in families with multiple cases of breast cancer, but have been associated with approximately 5 to 10 percent of ovarian cancers.

Possible treatments for ovarian cancer include surgery, immunotherapy, chemotherapy, hormone therapy, radiation therapy, or a combination thereof. Surgical procedures for the treatment of ovarian cancer include debulking, and a unilateral or bilateral oophorectomy and/or a unilateral or bilateral salpigectomy. Anti-cancer drugs that have also been used to treat ovarian cancer include cyclophosphamide, etoposide, altretamine, and ifosfamide. Hormone therapy with the drug tamoxifen is also used to shrink ovarian tumors. Radiation therapy optionally includes external beam radiation therapy and/or brachytherapy. The majority of newly-diagnosed ovarian cancer patients have been shown to respond to first-line platinum-based and paclitaxel chemotherapy. However, 50-80% of the patients who respond to this combination therapy will eventually relapse. See, e.g., Herzog, “Update on the role of topotecan in the treatment of recurrent ovarian cancer,” The Oncologist 7(Suppl. 5):3-10 (2002). Women with advanced ovarian cancer have poor long-term survival due to disease recurrence and most die within 5 years. Ovarian cancer relapsing within 6 months of platinum treatment represents a heterogeneous spectrum of disease with a low response rate to therapy (˜10%-25%), generally of short duration. Attempts to identify patients who will respond to specific drugs are challenging. There is clearly a need to improve the current treatment options for recurrent ovarian cancer.

The terms “heavily pre-treated recurrent ovarian cancer” and “platinum-resistant ovarian carcinoma”, as used herein, refer to ovarian cancer that has been treated with one or more rounds of platinum based chemotherapry using agents such as cisplatin, gemcitabine, carboplatin.

PD-L1 is expressed on many cancers including renal cell carcinoma, pancreatic cancer, ovarian cancer, gastric cancer, esophageal cancer, and hepatocellular carcinoma. It has been shown that PD-L1 expression on monocytes in the ascites and blood of patients with malignant ovarian cancer is strikingly higher than those with benign/borderline disease, with no overlap in the values between these groups. Furthermore, recent studies indicate that most ovarian cancers evade the host immune system and accelerate tumor growth by expressing PD-L1. Therefore, it is hypothesized that the PD-1/PD-L pathway may be a potential target for immunotherapy of ovarian cancer.

Methods for the Treatment of Recurrent Heavily Pre-Treated Recurrent Ovarian Cancer

One embodiment provides a method of treating ovarian cancer in a patient, wherein the method comprises, administering to the patient a combination comprising entinostat and an anti-PD-L1 antibody. Another embodiment provides the method, wherein the anti PD-L1 antibody is avelumab (MSB0010718C).

Another embodiment provides the method, wherein the cancer wherein the cancer is characterized by overexpression of PD-L1. Another embodiment provides the method, wherein the ovarian cancer is heavily pre-treated recurrent ovarian cancer. Another embodiment provides the method, wherein the heavily pre-treated recurrent ovarian cancer is epithelial ovarian carcinoma, fallopian tube cancer, or primary peritoneal carcinoma. Another embodiment provides the method, wherein the patient has received at least one round of a prior therapy. Another embodiment provides the method, wherein the prior therapy is platinum based chemotherapy.

Another embodiment provides the method, wherein the entinostat and anti-PD-L1 antibody are administered sequentially in either order or simultaneously. Another embodiment provides the method, wherein the anti-PD-L1 antibody is administered as intravenous infusion. Another embodiment provides the method, wherein the anti-PD-L1 antibody is administered once every two weeks at a dose of 10 mg/kg, by intravenous infusion. Another embodiment provides the method, wherein the entinostat is administered periodically during the treatment cycle. Another embodiment provides a method wherein the entinostat is administered orally. Another embodiment provides the method, wherein the entinostat is administered once every week during the treatment cycle, at a dose of 3 mg. Another embodiment provides the method, wherein the entinostat is administered once every week during the treatment cycle, at a dose of 5 mg. Another embodiment provides the method, wherein the entinostat is administered orally once every two weeks during the treatment cycle at a dose of 10 mg. Another embodiment provides the method, wherein entinostat is administered first. Another embodiment provides the method, wherein entinostat is administered first. Another embodiment provides the method, wherein entinostat is administered weekly. Another embodiment provides the method, wherein entinostat is administered every two weeks. Another embodiment provides the method, wherein the entinostat is administered every two weeks, at a dose of 5 mg. Another embodiment provides the method, wherein entinostat and anti-PD-L1 antibody are administered simultaneously.

### Additional Therapy

Available additional treatments for triple negative breast cancer that may be advantageously employed in combination with the therapies disclosed herein include, without limitation, radiation therapy, chemotherapy, antibody therapy, and tyrosine kinase inhibitors as adjuvant therapy.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

Different chemotherapeutic agents are known in the art for treating lung cancer. Cytoxic agents used for treating lung cancer include carboplatin (for example, Paraplatin®, Paraplat®), cisplatin (for example, Platinol®, Platinol-Aq®), crizotinib (for example Xalkori®), etoposide (for example Toposar®, VePesid®), etoposide Phosphate (for example Etopophos®), gemcitabine hydrochloride (for example Gemzar®), gemcitabine-cisplatin, methotrexate (for example Abitrexate®, Folex®, Folex Pfs®, Methotrexate Lpf®, Mexate®, Mexate-Aq®), paclitaxel (for example Taxol®), pemetrexed Disodium (for example Alimta®), and topotecan Hydrochloride (for example Hycamtin®)

Different agents are known in the art for treating melanoma, including aldesleukin (for example Proleukin®), dabrafenib (for example Tafinlar®), dacarbazine (for example DTIC-Dome®), recombinant Interferon Alfa-2b (for example Intron® A), Ipilimumab (for example Yervoy®), pembrolizumab (for example Keytruda®), Trametinib (for example Mekinist®), Nivolumab (for example Opdivo®), Peginterferon Alfa-2b (for example Pegintron®, Sylatron®), vemurafenib (for example Zelboraf®).

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

Additional, illustrative, treatments that may be advantageously combined with the compositions and therapies disclosed herein may include, without limitation, administration of agents including, but not limited to lapatinib, alone or in combination with capecitabine, docetaxel, epirubicin, epothilone A, B or D, goserelin acetate, paclitaxel, pamidronate, bevacizumab, or trastuzumab.

In some embodiments, the additional therapy comprises chemotherapy comprising administering to the subject one or more of doxorubicin, cyclophosphamide, paclitaxel, lapatinib, capecitabine, trastuzumab, bevacizumab, gemcitabine, eribulin, or nab-paclitaxel.

### Oral Formulations

Oral formulations containing the active pharmaceutical ingredients described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination therapy described herein can be given simultaneously or can be given in a staggered regimen, with entinostat being given at a different time during the course of chemotherapy than the EGFR inhibitor. This time differential may range from several minutes, hours, days, weeks, or longer between administrations of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non- effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action.

### EXAMPLES

### Example 1

A Phase 1B/2, open-label, study of entinostat in combination with avelumab (MSB0010718C) is carried out in patients with recurrent, heavily-pretreated ovarian cancer.

Entinostat has been shown in preclinical models to reduce the number of, and inhibit the function of, host immune suppressor cells in order to enhance the anti-tumor activity of immune checkpoint blockade. It is hypothesized that Entinostat combined with avelumab results in an improved overall response rate for the combination compared to either agent alone.

### Study Design:

The study is an open-label, Phase 1b/2 study evaluating the combination of entinostat plus avelumab in patients withrecurrent, heavily-pretreated ovarian cancer. The study has 2 phases: a Dose Escalation/Confirmation Phase (Phase 1b) and an Expansion Phase (Phase 2), with the Expansion Phase utilizing a Simon 2-stage design for each cohort.

Phase 1B (Dose Escalation Phase)

### Objectives:

### Dose

Determine the dose-limiting toxicities (DLT) and maximum tolerated dose (MTD) or recommended Phase 2 dose (RP2D) of entinostat (SNDX-275) given in combination with avelumab.

### Safety

Evaluate safety and the tolerability of entinostat in combination with avelumab, as measured by clinical adverse events (AEs) and laboratory parameters.

The starting dose (dose level 1) for entinostat is 5 mg by mouth (po) weekly. The dose of avelumab is fixed at 10 mg/kg IV infusion every two weeks (Q2W) for all cohorts.

If dose level 1 is not tolerated, dose level −1 for entinostat is set at 3 mg po weekly.

Each dose level in the dose escalation phase enrolls between 6 and 12 evaluable patients.

TABLE 1
Dose Escalation Schematic

| | Number of | | |
|---|---|---|---|
| Cohort | Subjects | Entinostat Dose | Avelumab Dose Q2W |
| −1 | 6-12 | 3 mg po QW | 10 mg/kg IV infusion |
| 1 | 6-12 | 5 mg po QW | 10 mg/kg IV infusion |

### Safety

Safety is assessed during the study by documentation of AEs, clinical laboratory tests, physical examination, vital sign measurements, electrocardiograms (ECGs), and Eastern Cooperative Oncology Group (ECOG) performance status.

Any detected cumulative toxicity may require later dose reductions and/or other changes to the dosing schedule, as appropriate, including further refinement of the RP2D.

If the 5 mg dose exceeds the MTD, then a 3 mg dose is evaluated. Toxicities are assessed by the study Investigator using the United States (US) National Cancer Institute (NCI) Common Terminology Criteria for Adverse Events (CTCAE), version 4.03. The decision regarding whether to proceed to the next dose level is made by the Medical Monitor in consultation with the study Investigators after the majority of the safety assessments for each cohort are completed.

After completion of the Dose Escalation/Confirmation Phase of the study, with identification of the MTD/RP2D, the Phase 2 portion of the study commences.

### Phase 2

Phase 2 (Expansion): In the Expansion Phase, entinostat in combination with avelumab is evaluated using the RP2D identified in the Dose Escalation/Confirmation Phase in women with heavily pre-treated ovarian cancer. In the phase 2 component, women with heavily pre-treated ovarian cancer are randomized in a 2:1 ratio to receive avelumab plus entinostat (at the RP2D) or avelumab plus placebo. The primary endpoint of this randomized component is progression free survival (PFS) as assessed by irRECIST. Secondary endpoints includes overall response rate (ORR), overall survival (OS), and safety. The sample size in the Phase 2 component is 120 patients, which results in 90% power with a one-sided p=0.1 to detect a PFS benefit with a hazard ratio (HR)=0.57. The duration of treatment for the test arm is 7 months and control arm is 4 months. Accrual is estimated to take 12 months, and the duration of the trial is projected to be 2 years.

An early look using overall response (OR), as well as OR+standard deviation at 16 weeks is implemented after a prospectively defined number of patients have been followed for a prospectively defined number of weeks, as mutually agreed by the Joint Development Committee.

### Summary of Patient Eligibility Criteria:

### Inclusion Criteria

Patients with recurrent, heavily-pretreated (3 or more prior regimens) ovarian cancer are eligible to participate in the study.

### Example 2

### Modulation of Antibody-Dependent Cell-Mediated Cytotoxicity (ADCC) Mediated by the Anti-PD-L1 Antibody Avelumab on Human Lung and Prostate Carcinoma Cell Lines Using the HDAC Inhibitors Vorinostat and Entinostat.

Checkpoint inhibitors targeting the PD-1/PD-L1 axis are promising immunotherapies shown to elicit objective responses against multiple tumor types. Yet despite their clinical promise, these agents fail to benefit most patients with solid carcinomas. Although epigenetic therapies have shown limited clinical benefit for patients with solid malignancies, histone deacetylase (HDAC) inhibitors have been shown to delete immunosuppressive elements and promote synergistic antitumor effects in combination with various immunotherapies. Aberrant HDAC expression has also been associated with poor prognosis in several cancer types. In this Example, the potential of clinically relevant exposure of lung and prostate carcinoma cells to HDAC inhibitors was examined, to increase natural killer tumor lysis mediated by avelumab, a fully human IgG1 monoclonal antibody (mAb) targeting PD-L1. Statistical significance relative to controls (2-way ANOVA). * p<0.05; ** p<0.01; *** p<0.001.

Drug Exposure, Phenotype & Killing Assays

Carcinoma cells were exposed daily for 5 h to vorinostat (3 μM) or DMSO for 4 consecutive days, or to entinostat (500 nM) or DMSO for 72 h, prior to being examined for (a) cell-surface PDL1 expression by flow cytometry or (b) used as target cells in a 4 h ¹¹¹In release assay where NK cells purified from 2 from healthy donors were used as effectors (E:T=30:1).

ADCC

NK lysis was performed in the presence of avelumab or isotype control (2 ng/mL).

CD16 Blocking

NK cells were pretreated for 2 h with anti-CD16 blocking antibody (12 μg/mL) prior to being used as effectors.

Table 2 below shows the effect of HDAC inhibitors on tumor viability and cell-surface expression of PD-L1, MICA/B, and HLA-ABC. Carcinoma cells were exposed to vorinostat, entinostat, or DMSO, prior to flow cytometric analysis. Values in bold represent an increase of 25% in protein levels and/or MFI in treated cells compared to controls.

TABLE 2

| | Tumor | Surface | % Positive (MFI) | | | |
|---|---|---|---|---|---|---|
| Cell line | type | molecule | DMSO | Vorinostat | DMSO | Entinostat |
| DU 145 | Prostate | PD-L1 | 76.3 (200) | 96.0 (354) | 81.4 (226) | 99.5 (705) |
| | | MIC-A/B | 42.9 (118) | 93.9 (371) | 58.9 (149) | 94.3 (447) |
| | | HLA-ABC | 100 (200) | 100 (354) | 100 (226) | 100 (705) |
| | | Viability | 98.2% | 96.4% | 98.7% | 97.9% |
| PC-3 | Prostate | PD-L1 | 74.1 (207) | 83.3 (251) | 55.8 (146) | 86.4 (240) |
| | | MIC-A/B | 33.2 (102) | 58.1 (137) | 43.7 (117) | 91.5 (268) |
| | | HLA-ABC | 99.8 (699) | 99.7 (816) | 99.9 (801) | 100 (1144) |
| | | Viability | 98.9% | 99% | 99.2% | 97.8% |
| NCI-H460 | Lung | PD-L1 | 68.0 (199) | 86.9 (278) | 89.5 (492) | 90.2 (507) |
| | | MIC-A/B | 4.4 (45) | 70.6 (213) | 20.3 (76) | 69.8 (189) |
| | | HLA-ABC | 86.6 (371) | 97.1 (608) | 99.2 (822) | 94.3 (547) |
| | | Viability | 99% | 97.7% | 99.1% | 96.5% |
| NCI-H441 | Lung | PD-L1 | 99.6 (958) | 99.8 (1053) | 99.7 (1031) | 99.7 (1027) |
| | | MIC-A/B | 23.5 (116) | 21.1 (111) | 16.7 (101) | 46.2 (172) |
| | | HLA-ABC | 99.9 (1182) | 99.9 (1284) | 99.9 (1232) | 99.9 (1375) |
| | | Viability | >95% | >95% | >95% | >95% |
| AsPC-1 | Pancreas | PD-L1 | 1.4 (87.5) | 1.4 (90.4) | 1.9 (91.4) | 9.9 (126) |
| | | MIC-A/B | 1.9 (88.3) | 0.8 (78.2) | 1.4 (76) | 6.5 (106) |
| | | HLA-ABC | 98.5 (625) | 99.2 (806) | 99.1 (670) | 99.0 (126) |
| | | Viability | 97.5% | 96.9% | 97.3% | 95.6% |

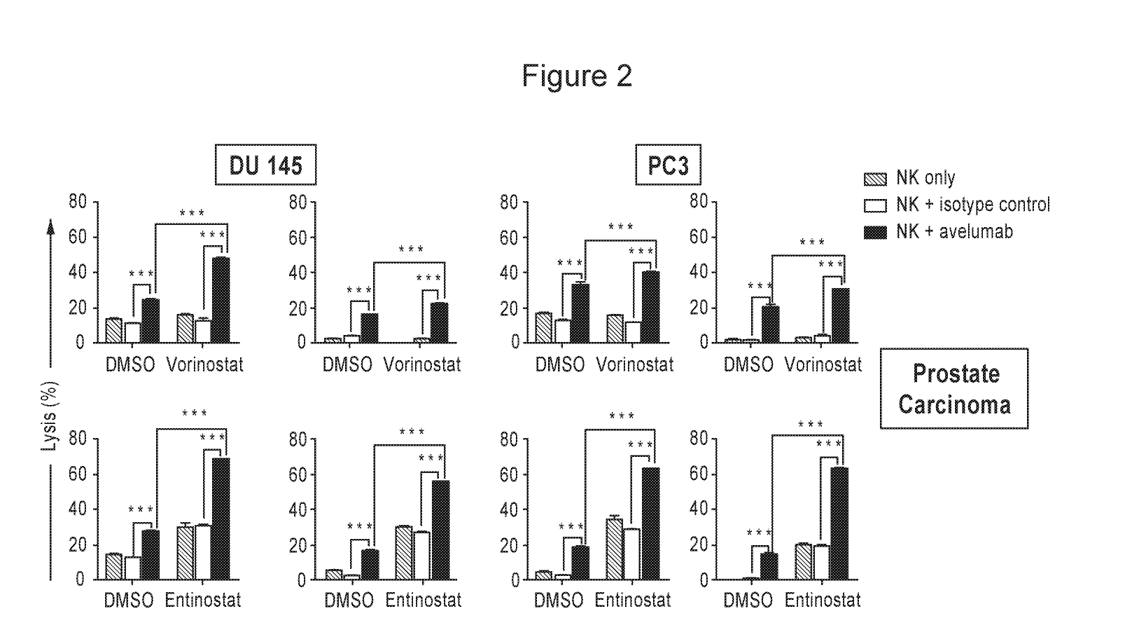

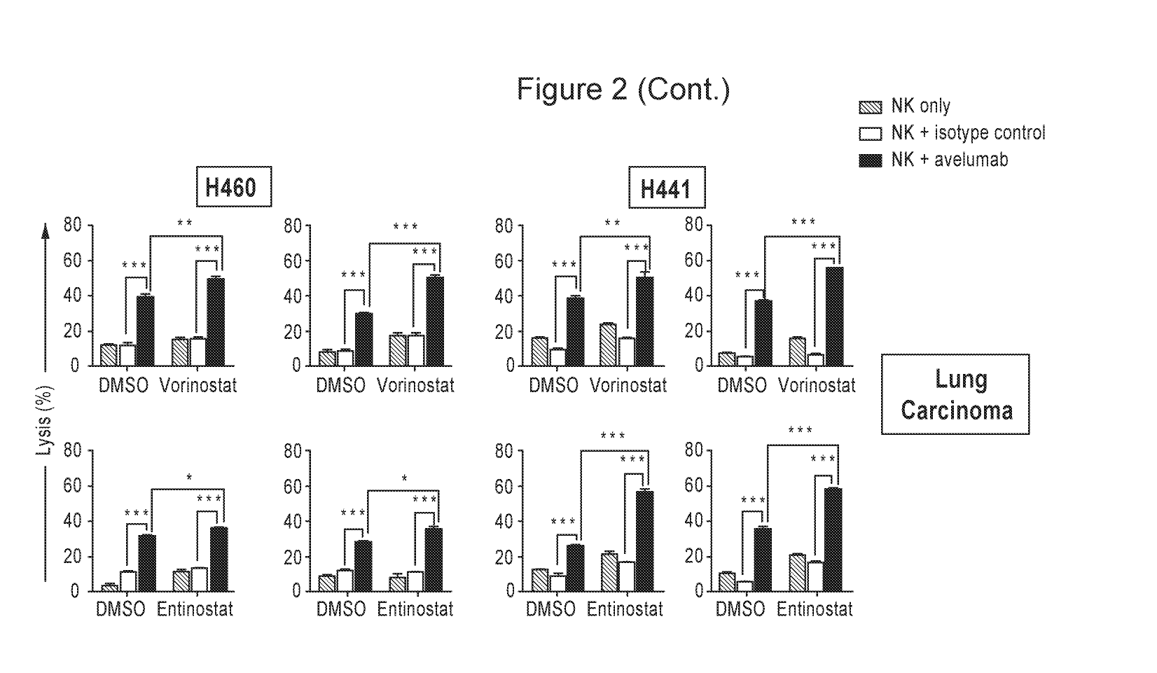

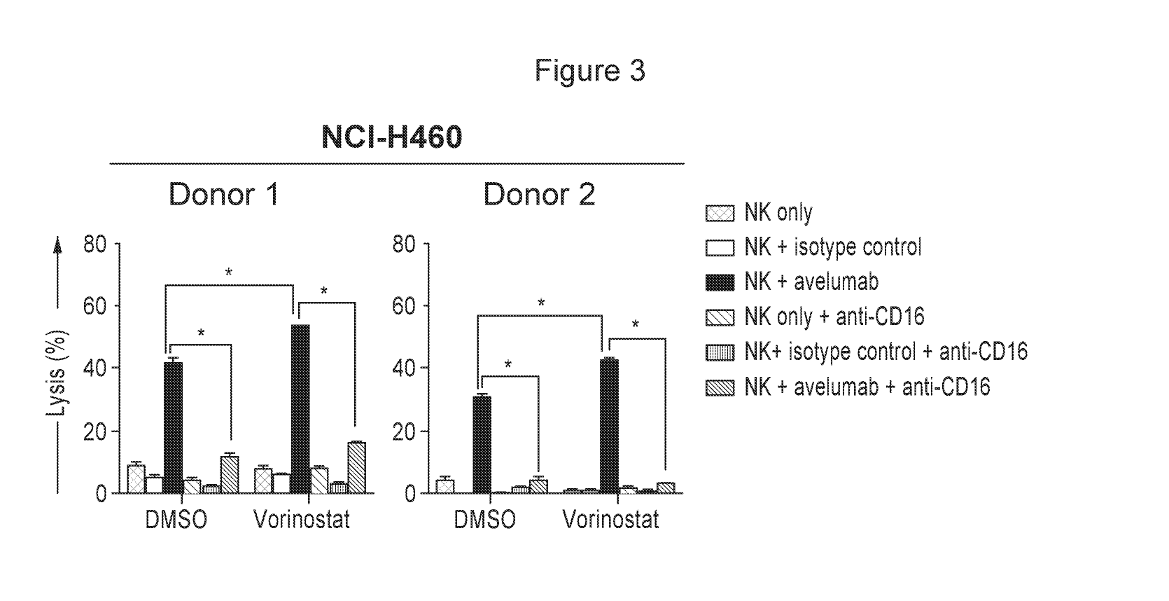

Further, FIG. 2 demonstrates that lung and prostate carcinoma cells are more sensitive to avelumab-mediated antibody-dependent cytotoxicity (ADCC) in vitro after clinically relevant epigenetic priming with either the pan-HDAC inhibitor vorinostat or the class I HDAC inhibitor entinostat. DU 145, PC-3, NCI-H460, and NCI-H441 carcinoma cells were exposed to vorinostat, entinostat, or DMSO prior to being used as targets for NK-cell lysis in the presence or absence of avelumab or isotype control. Results are presented as mean±S.E.M. from 3 replicate wells, and are representative of 2-4 independent experiments. FIG. 3 shows that avelumab-mediated lysis of carcinoma cells is decreased by CD16 neutralization. NCI-H460 lung carcinoma cells were exposed to vorinostat or DMSO, prior to being used as targets for NK-cell lysis in the presence or absence of avelumab or isotype control. For CD16 blocking, NK cells were pretreated for 2 h with anti-CD16 blocking antibody prior to being used as effectors. Results are presented as mean±S.E.M. from 3 replicate wells, and are representative of 2-4 independent experiments. Importantly, the results indicate that augmented avelumab-mediated ADCC of tumor targets exposed to HDAC inhibitors can occur without altering tumor PD-L1 expression.

Human Xenografts & PD-L1 Immunofluorescence

Female nu/nu mice were implanted with NCI-H460 (lung) or PC-3 (prostate) carcinoma cells. When tumors reached 0.5-1 cm³, animals received 4 daily doses of DMSO or vorinostat (150 mg/kg, p.o.). Alternatively, animals received a single dose of entinostat (20 mg/kg, p.o.) or DMSO 72 h prior to tumor excision. Frozen specimens were examined for cell-surface expression of PD-L1 by immunofluorescence using antihuman PD-L1 (clone SP142) and goat anti-rabbit AF594, and counterstained with DAPI-containing mounting medium.

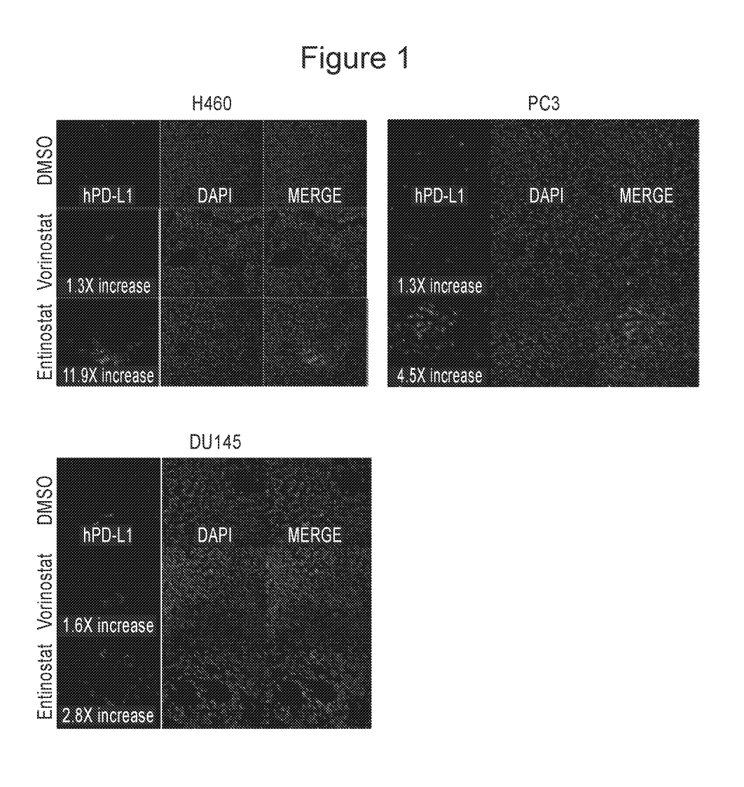

FIG. 1 shows that both HDAC inhibitors (i.e., vorinostat and entinostat) can enhance PD-L1 expression in vivo in lung and prostate carcinoma xenografts. Female nu/nu mice implanted with NCI-H460, PC-3, or DU 145 carcinoma cells received 4 daily doses of DMSO or vorinostat (150 mg/kg, p.o.). Tumors were excised 24 h after the last dose. Alternatively, animals received a single dose of entinostat (20 mg/kg, p.o.) or DMSO 72 h prior to tumor excision. Frozen sections were examined for cell-surface expression of human PD-L1 by immunofluorescence. Confocal images are shown at 20× magnification and are representative of 3 animals/treatment. Relative PD-L1 expression levels were calculated using ImageJ software by normalizing the intensity values to their respective DMSO treated controls.

In summary, vorinostat and entinostat significantly increased sensitivity of human lung and prostate carcinoma cells to ADCC mediated by avelumab. The anti-CD16 neutralizing mAb significantly decreases avelumab-mediated lysis of target cells exposed to either HDAC inhibitor. Both HDAC inhibitors can enhance tumor PD-L1 expression in vitro and in vivo in prostate and/or lung xenograft models. Increased avelumab-mediated ADCC of tumor targets exposed to HDAC inhibitors can occur without increased tumor PD-L1 expression. These studies provide a rationale for combining vorinostat or entinostat with monoclonal antibodies targeting PD-L1, including for patients that have failed monotherapy regimens with HDAC or checkpoint inhibitors.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating cancer, comprising administering to a patient a combination comprising entinostat and an anti-PD-L1 antibody, wherein the cancer is ovarian cancer.

2. The method of claim 1, wherein the anti PD-L1 antibody is avelumab.

3. The method of claim 1, wherein the ovarian cancer is heavily pre-treated recurrent ovarian cancer.

4. The method of claim 3, wherein the heavily pre-treated recurrent ovarian cancer is epithelial ovarian carcinoma, fallopian tube cancer, or primary peritoneal carcinoma.

5. The method of claim 4, wherein the heavily pre-treated recurrent ovarian cancer is epithelial ovarian cancer.

6. The method of claim 1, wherein the patient has received at least one round of a prior therapy.

7. The method of claim 1, wherein the patient has received at least three rounds of a prior therapy.

8. The method of claim 6, wherein the prior therapy is platinum based chemotherapy.

9. The method of claim 8, wherein the patient has a relapse of ovarian cancer within six months after the last round of platinum based chemotherapy.

10. The method of claim 1, wherein entinostat and anti-PD-L1 antibody are administered sequentially in either order or simultaneously.

11. (canceled)

12. The method of claim 1, wherein the anti-PD-L1 antibody is administered once every two weeks during the treatment cycle, at a dose of 10 mg/kg.

13. The method of claim 1, wherein the entinostat is administered orally.

14. The method of claim 13, wherein the entinostat is administered once every week during the treatment cycle, at a dose of 3 mg.

15. The method of claim 13, wherein the entinostat is administered once every week during the treatment cycle, at a dose of 5 mg.

16. The method of claim 13, wherein the entinostat is administered once every two weeks during the treatment cycle, at a dose of 10 mg.

17. The method of claim 1, wherein entinostat is administered first.

18. The method of claim 1, wherein the entinostat is administered weekly.

19. The method of claim 1, wherein the entinostat is administered every two weeks.

20. The method of claim 19, wherein the entinostat is administered at a dose of 5 mg.

21. The method of claim 1, wherein entinostat and anti-PD-L1 antibody are administered simultaneously.

22. A kit for treating heavily pre-treated recurrent ovarian cancer comprising a combination of entinostat and an anti-PD-L1 antibody.

23. The kit of claim 22, wherein the anti-PD-L1 antibody is avelumab.

24. The method of claim 7, wherein the prior therapy is platinum-based chemotherapy.
